(19) United States
(12) Patent Application Publication
(10) Pub. No.: US 2003/0134804 A1
(43) Pub. Date: Jul. 17, 2003

(54) Rehydrating formulation

(76) Inventors: Roderick Frederick Gerardus Joseph King, Otley (GB); Simon Edmund George Lester, London (GB)

Correspondence Address: Sullivan Law Group, Suite 1140, 1850 North Central Avenue, Phoenix, AZ, 85004-4586, US

(21) Appl. No.: 10/289,240

(22) Filed: Nov. 6, 2002

Related U.S. Application Data
(60) Provisional application No. 60/340,751, filed on Nov. 30, 2001.

(30) Foreign Application Priority Data
Nov. 7, 2001 (GB) GB 0126746.7

(51) Int. Cl. A61K 31/70; A61K 33/14

(52) U.S. Cl. 514/23; 424/680

(57) ABSTRACT

The present invention relates to an aqueous formulation for combatting dehydration comprising a low concentration of galactose and a source of sodium ions which is particularly effective in children (e.g., infants). The dehydration is typically a symptom of severe diarrhoea.

### RELATED APPLICATIONS

This application claims the benefit of priority of U.S. provisional patent application 60/340,751 filed in the U.S. Patent & Trademark Office on Oct. 31, 2001, the disclosure of which is incorporated herein by reference.

This application further claims the benefit of priority of Great Britain patent application GB 0126746.7 filed in the United Kingdom Patent Office on Nov. 7, 2001, the disclosure of which is incorporated herein by reference.

### BACKGROUND OF THE INVENTION

1. Field of the Invention

The present invention relates to an aqueous formulation for combatting dehydration comprising a low concentration of galactose and a source of sodium ions (e.g., a sodium salt).

2. Description of the Related Art

There are a number of circumstances in which a subject may suffer dehydration and associated deficiencies such as loss of mineral salts. For example, exercise acts to deplete the body of fuel stores and increases the rate of perspiration causing loss of water and mineral salts. These losses can be significant if exercise is prolonged and particularly if ambient temperatures are moderate or high. Other circumstances which may lead to dehydration include excessive temperatures, nutritional deprivation, fever, diarrhoea and vomiting excess as a result of (for example) enterotoxin effects (e.g., cholera typically in the third world), viral effects (e.g., rotavirus typically in the developed world) or post surgical rest (e.g., when GI rest is important). Alternatively, the circumstances which may lead to dehydration may be self-induced (e.g., through alcohol consumption). In extreme cases, loss of fluid and/or electrolytes may be clinically important or even life threatening, in particular in children and the elderly.

Humans do not function efficiently when dehydrated or when they are suffering an electrolyte imbalance. Rehydrating drinks containing inorganic salts and/or high sugar (e.g., glucose) content are well established in combatting these disorders. However the administration of glucose may lead to exaggerated insulin responses.

Galactose is a naturally occurring hexose for which worldwide demand is negligible and reported uses scarce. Prior publications relating to galactose include:

(1) WO-A-01/28360 (Marathade Ltd) discloses high energy multi-saccharide food products containing galactose and creatine for use in sport or to combat hunger or fatigue;

(2) EP-A-0340491(Biodyn Ag) discloses a foodstuff in which the saccharide component is primarily galactose;

(3) WO-A-96/18313 (University of Nottingham) discloses formulations for increasing creatine uptake comprising creatine, insulin and a simple carbohydrate such as galactose;

(4) WO-A-98/06418 (Mannatech, Inc) discloses dietary supplements comprising galactose for nutritional support and treating various disorders;

(5) US-A-5843921 (Childrens Hospital of Los Angeles) discloses formulations for treatment of diabetes comprising less than 3 g per unit of simple sugars including galactose;

(6) WO-A-96/08979 (Quadrant Holdings Cambridge Ltd) discloses sports beverages comprising trehalose and galactose;

(7) WO-A-90/02494 (Svenska Mejeriernas Riksforening Ekonomi AB) discloses a sports drink comprising galactose originating from a desalinated and hydrolised whey concentrate;

(8) EP-A-349712 (Biodyn Ag) discloses foodstuffs containing a tooth protecting amount of galactose in the form of lactose hydrosylate; and\

(9) EP-A-184121 (Biodyn Ag) discloses anti-caries additives for sucrose foodstuffs comprising galactose I.

U.S. Pat. No. 5,780,094 (Marathade Limited) discloses a rehydrating sports drink containing a high concentration of galactose and a proportion of glucose. The absorption of water in the intestine is efficient and effective when driven by sodium co-transport involving a high concentration of galactose and glucose. Galactose advantageously effects more rapid intestinal uptake of water than does glucose.

The present invention is based on the recognition that being physiologically adapted to effectively metabolise galactose and glucose (the two constituent sugars of lactose in milk) children are able to exploit sodium co-transport for effective rehydration using a low concentration of galactose. In particular, the present invention relates to an improved rehydrating aqueous formulation comprising a low concentration of galactose and a sodium salt to combat dehydration resulting from (for example) diarrhoea.

Thus viewed from one aspect the present invention provides an aqueous formulation for combatting dehydration comprising galactose at a concentration in the range 25 to 135 mM, a source of sodium ions and water.

The aqueous formulation of the invention is generally more effective than a conventional glucose-containing rehydrating solution in treating extreme dehydration which might otherwise be fatal to a child. Moreover galactose (unlike glucose) does not elicit a primary insulin response.

In a preferred embodiment, the concentration of galactose in the aqueous formulation is in the range 25 to 130 mM, preferably 37 to 120 mM, more preferably 40 to 120 mM, especially preferably 45 to 90 mM.

The source of sodium ions in the aqueous formulation of the invention is typically a sodium salt. Any physiologically tolerable sodium salt will suffice. Examples include sodium lactate, sodium chloride, sodium citrate, trisodium citrate, sodium hydrogenphosphate, disodium hydrogen phosphate and sodium bicarbonate. The counter ion (e.g., chloride, bicarbonate, phosphate, hydrogenphosphate or citrate) may provide stability and buffering capacity and sodium citrate is advantageous for acidosis associated with diarrhoea of specific aetiology (e.g., viral). Sodium chloride is preferred.

The concentration of sodium ions may be adapted to facilitate sodium co-transport and replacement of electrolyte loss. In a preferred embodiment, the concentration of sodium ions in the aqueous formulation is in the range 25 to 100 mM, preferably 30 to 90 mM, particularly preferably 35 to 90 mM, more preferably 45 to 90 mM, yet more preferably 60 to 90 mM. For combatting dehydration as a symptom of severe diarrhoea (e.g., caused by enterotoxin effects), higher sodium concentrations are preferred (eg 80-90 mM such as about 90 mM). For combatting dehydration as a symptom of less severe diarrhoea (e.g., caused by viral effects or when hypernatraemia is a risk), lower sodium concentrations are preferred.

An embodiment of the aqueous formulation further comprises one or more sources of additional ions (e.g., potassium, magnesium, calcium or zinc) to advantageously achieve replacement of minerals which have been lost as a symptom of (for example) diarrhoea. The source of an additional ion is typically a salt (e.g., a mineral salt) such as a chloride.

Preferably the aqueous formulation comprises a source of potassium ions (eg a potassium salt such as potassium chloride). The potassium salt serves to replace electrolyte loss. In a preferred embodiment, the concentration of potassium ions in the aqueous formulation is in the range 5 to 35 mM, preferably 10 to 30 mM, particularly preferably 15 to 25 mM, more preferably about 20 to 25 mM.

An embodiment of the aqueous formulation of the invention further comprises one or more additional carbohydrates.

In an embodiment of the invention, the one or more additional carbohydrates may be a digestible saccharide such as a digestible saccharide selected from one or more of the group consisting of monosaccharides, disaccharides, oligosaccharides and polysaccharides. These may be natural or synthetic saccharides. For example, the one or more additional carbohydrates may be selected from the group consisting of glucose, sucrose, dextrose, fructose, lactose and maltose. Preferably the additional carbohydrate is a monosaccharide, particularly preferably glucose. In situations where the osmolality of the aqueous formulation is important (e.g., where hyperosmolar or isomolar solutions are contra indicated), maltodextrin or higher oligosaccharide may be used in place of glucose.

In a preferred embodiment, the galactose and optionally one or more additional carbohydrates of the aqueous formulation are present in a total amount sufficient to meet the carbohydrate requirements of sodium co-transport for effective rehydration. In a preferred embodiment, the total concentration of carbohydrate is in the range 50 to 300 mM, preferably 50 to 200 mM, particularly preferably 55 to 175 mM, more preferably 60 to 135 mM, especially preferably 90 to 130 mM.

In a preferred embodiment of the formulation, galactose and glucose are present in a total amount sufficient to meet the carbohydrate requirements of sodium co-transport in effective rehydration. Typically the ratio of molar concentration of galactose and glucose is in the range 0.6:1 to 1:0.6, preferably 0.8:1 to 1:0.8, particularly preferably 0.9:1 to 1:0.9, more preferably the molar concentration ratio is about 1:1.

In a preferred embodiment, the concentration of glucose in the aqueous formulation is in the range 30 to 135 mM, preferably 35 to 130 mM, particularly preferably 37 to 120 mM, more preferably 40 to 100 mM, especially preferably 45 to 80 mM.

In situations where the osmolality of the aqueous formulation is important (e.g., where hyperosmolar or isomolar solutions are contra indicated), maltodextrin or higher oligosaccharide is preferably used in place of glucose. Thus the ranges of preferred glucose concentration may be satisfied by a concentration of an oligosaccharide with an appropriate chain length. For example, in place of glucose at 60 mM, a maltodextrin of average chain length six could be used at 10 mM.

A general purpose formulation might typically have a ratio of sodium ion concentration:carbohydrate concentration of about 1:2 eg a concentration of sodium ions of about 60 mM and galactose and glucose present at a total concentration of 120 mM. Such a formulation would be useful where the circumstance leading to dehydration is diarrhoea as a result of post surgical rest (e.g., when GI rest is important) or the circumstance leading to dehydration is self-induced (e.g., through alcohol consumption).

The aqueous formulation of the invention may be administered by any convenient route. Preferably the aqueous formulation is adapted for oral administration. For example, the aqueous formulation may be palatable and for this purpose may further comprise natural or synthetic flavourings such as fruit flavourings (e.g., blackcurrant, strawberry, apple, citrus, lemon, lime, orange or cranberry) or caffeine and sweeteners.

The aqueous formulation of the invention may further comprise physiologically tolerable stabilisers, anti-oxidants (e.g., ascorbic acid) and preservatives (e.g., sodium benzoate or sorbic acid) as desired.

The administration dosage generally depends on the level of dehydration and the size and age of the subject. Generally it is advisable for the dose to be equivalent to or slightly greater than the actual or expected loss of bodily water and to be administered at appropriate intervals. Typically a dose of aqueous formulation is in the range 100 to 250 ml.

Citric acid may be added to the aqueous formulation for partial or total replacement of citrate ion and for buffering purposes (typically to maintain pH in the range 2 to 6). Where the source of sodium or other ions is a citrate salt and/or citric acid is added, the concentration of citrate ion in the aqueous formulation may be in the range 5 to 30 mM, preferably 10 to 25 mM, particularly preferably 10 to 15 mM. A phosphate salt may be used as an alternative buffering agent.

Where the source of sodium (and optionally additional ions) is a chloride salt, the concentration of chloride ion in the aqueous formulation may be in the range 10 to 100 mM, preferably 30 to 90 mM, more preferably 40 to 85 mM, especially preferably 45 to 80 mM. For combatting dehydration as a symptom of severe diarrhoea, higher chloride concentrations are preferred (e.g., 70-80 mM such as about 80 mM).

The aqueous formulation of the invention may be an aqueous solution, aqueous dispersion or aqueous suspension. Preferably the aqueous formulation is an aqueous solution. Preferably the aqueous formulation (e.g., solution) is a reconstituent aqueous formulation (e.g., solution). For example, a reconstituent aqueous formulation of the invention may be reconstituted by the end user at the point of use from a composition comprising galactose and a source of sodium ions (and optionally one or more sources of additional ions and one or more additional carbohydrates) by addition of a suitable volume of aqueous solvent (e.g., water).

Viewed from a further aspect the present invention provides a composition formulable (e.g., dissolvable) in water to form an aqueous formulation as hereinbefore defined, said composition comprising galactose, a source of sodium ions, optionally an additional carbohydrate and optionally a source of additional ions. For example, the composition comprises galactose in an amount sufficient to form a concentration in the range 25 to 135 mM in a specified volume of aqueous solvent (e.g., water) and a source of sodium ions.

The composition of the invention may be provided in any suitable solid or liquid form. For example, the composition may be provided in solid form such as powdered (e.g., effervescent or non-effervescent powdered) or tablet form or in liquid form such as gel or liquid concentrate form.

Viewed from a yet further aspect the present invention provides the use of galactose and a source of sodium ions for the preparation of (A) an aqueous 25 to 135 mM galactose formulation for combatting (e.g., treating or preventing) dehydration or (B) a medicament formulable (e.g., dissolvable in water) into an aqueous 25 to 135 mM galactose formulation for combatting (e.g., treating or preventing) dehydration.

In a preferred embodiment of the use of the invention, the aqueous 25 to 135 mM galactose formulation is as hereinbefore defined.

In a preferred embodiment of the use of the invention, the medicament is a composition as hereinbefore defined.

In a preferred embodiment, the present invention provides the use of galactose and a source of sodium ions for the preparation of (A) an aqueous 25 to 135 mM galactose formulation for combatting dehydration and associated deficiencies or (B) a medicament formulable into an aqueous 25 to 135 mM galactose formulation for combatting dehydration and associated deficiencies. The associated deficiencies may be mineral loss, electrolyte imbalance, etc.

In a preferred embodiment, the present invention provides the use of galactose and a source of sodium ions for the preparation of (A) an aqueous 25 to 135 mM galactose formulation for combatting dehydration in children (e.g., infants) or (B) a medicament formulable into an aqueous 25 to 135 mM galactose formulation for combatting dehydration in children (e.g., infants).

Preferably the dehydration and (where present) associated deficiencies are symptoms of diarrhoea. The diarrhoea may be a result of (for example) enterotoxin effects (e.g., cholera typically in the third world), viral effects (e.g., rotavirus typically in the developed world) or post surgical rest (e.g., when GI rest is important). The concentration of each component may be tailored to optimise treatment of dehydration and associated deficiencies caused by mild, moderate or severe diarrhoea. For combatting dehydration as a symptom of severe diarrhoea, higher sodium and chloride concentrations are preferred. Alternatively, the circumstances which may lead to dehydration may be self-induced (e.g., through alcohol consumption).

In a preferred embodiment of the use of the invention, the dehydration or associated deficiencies are life threatening.

Viewed from a still yet further aspect the present invention provides a method for combatting dehydration in a subject comprising the step of: administering an effective dose of an aqueous formulation as hereinbefore defined to the subject.

Viewed from an even still further aspect the present invention provides a method for combatting dehydration and associated deficiencies in a subject comprising the step of administering an effective dose of an aqueous formulation as hereinbefore defined to the subject.

Although the subject may be any age, preferably the subject is a child (e.g., an infant). Preferably the dehydration and (where present) associated deficiencies are symptoms of diarrhoea (e.g., severe diarrhoea). The diarrhoea may be a result of (for example) enterotoxin effects (e.g., cholera typically in the third world), viral effects (e.g., rotavirus typically in the developed world) or post surgical rest (e.g., when GI rest is important). Alternatively, the circumstances which may lead to dehydration may be self-induced (e.g., through alcohol consumption).

The present invention will now be described in a non-limitative sense with reference to the following Example.

### EXAMPLE

Nine reconstituent aqueous formulations A, A1 and B-H were prepared which had the components and concentrations after reconstitution in water shown in Table 1:

TABLE 1

| | Concentration mM | | | | | | | |
|---|---|---|---|---|---|---|---|---|
| Substance | A(A1) | B | C | D | E | F | G | H |
| Galactose | 45(90) | 60 | 80 | 40 | 45 | 45 | 75 | 55 |
| Glucose | 45(0) | 60 | 80 | 40 | 45 | 45 | 75 | 55 |
| Sodium | 45 | 70 | 25 | 30 | 35 | 20 | 25 | 60 |
| Chloride | | | | | | | | |
| Potassium | 25 | 20 | 20 | 15 | 20 | 25 | 20 | 20 |
| Chloride | | | | | | | | |
| Trisodium | 10 | 10 | 10 | 10 | 15 | 13.3/ | 0 | 10 |
| Citrate/ | | | | | | 6.7 | | |
| Citric Acid | | | | | | | | |
| Sucrose | 0 | 0 | 0 | 80 | 20 | 0 | 0 | 0 |
| Fructose | 0 | 0 | 0 | 1 | 2 | 0 | 0 | 0 |
| Sodium | 0 | 0 | 0 | 0 | 0 | 0 | 15 | 0 |
| bicarbonate | | | | | | | | |

A solid mixture of pre-weighed components was prepared such that the specified concentrations were obtained when a certain proportion was dissolved in a specified volume of water. The unit volume of water added to reconstitute the composition may be up to 1000 ml. Typically 250 ml would be appropriate and a single dose may be made up of one, two or three 250 ml unit volumes administered at appropriate intervals. Each aqueous formulation may be flavoured to be palatable as desired using lemon, lime, blackcurrant, orange, citrus or cranberry.

The sodium concentration for compositions F, G and H are 60, 40 and 90 mM respectively and the chloride concentrations are 45, 45 and 80 mM respectively. Higher sodium and chloride concentrations are desirable for effective treatment of severe diarrhoea. Thus compositions F, G and H are useful in the treatment of moderate, mild and severe diarrhoea respectively.

General purpose formulations I, J, K, L, M and N are shown in Table 2.

TABLE 2

Concentration mM

| Substance | I | J | K | L | M | N |
|---|---|---|---|---|---|---|
| Galactose | 120 | 120 | 60 | 60 | 60 | 60 |
| Glucose | — | — | 60 | 60 | — | — |
| Sodium | 60 | 30 | 60 | 30 | 60 | 30 |
| Chloride | | | | | | |
| Potassium | 20 | 20 | 20 | 20 | 20 | 20 |
| Chloride | | | | | | |
| Trisodium | — | 13.3/ | — | 13.3/ | — | 13.3/ |
| Citrate/ | | 6.7 | | 6.7 | | 6.7 |
| Citric Acid | | | | | | |
| Maltodextrin | 0 | 0 | 0 | 0 | 10 | 10 |
| (chain 6) | | | | | | |

What is claimed is:

1. An aqueous formulation for combatting dehydration comprising galactose at a concentration in the range 25 to 135 mM, a source of sodium ions, and water.

2. An aqueous formulation as claimed in claim 1, wherein the concentration of galactose in the aqueous formulation is in the range 25 to 130 mM.

3. An aqueous formulation as claimed in claim 1, wherein the concentration of galactose in the aqueous formulation is in the range 37 to 120 Mm.

4. An aqueous formulation as claimed in claim 1, wherein the concentration of galactose in the aqueous formulation is in the range 40 to 120 mM.

5. An aqueous formulation as claimed in claim 1, wherein the source of sodium ions comprises sodium chloride optionally together with sodium citrate.

6. An aqueous formulation as claimed in claim 1, wherein sodium ions are present at a concentration in the aqueous formulation in the range 25 to 100 mM.

7. An aqueous formulation as claimed in claim 1, wherein sodium ions are present at a concentration in the aqueous formulation in the range 30 to 90 mM.

8. An aqueous formulation as claimed in claim 1, wherein sodium ions are present at a concentration in the aqueous formulation in the range 35 to 90 mM.

9. An aqueous formulation as claimed in claim 1, wherein sodium ions are present at a concentration in the aqueous formulation in the range 45 to 90 mM.

10. An aqueous formulation as claimed in claim 1, wherein sodium ions are present at a concentration in the aqueous formulation in the range 60 to 90 mM.

11. An aqueous formulation as claimed in claim 1, further comprising a source of potassium ions at a concentration in the aqueous formulation in the range 20 to 25 mM.

12. An aqueous formulation as claimed in claim 1, further comprising one or more additional carbohydrates selected from the group consisting of glucose, sucrose, dextrose, fructose, lactose and maltose.

13. An aqueous formulation as claimed in claim 1, wherein the one or more additional carbohydrates are present at a total concentration in the aqueous formulation in the range 50 to 300 mM.

14. An aqueous formulation as claimed in claim 1, wherein the one or more additional carbohydrates are present at a total concentration in the aqueous formulation in the range 50 to 200 mM.

15. An aqueous formulation as claimed in claim 1, wherein the one or more additional carbohydrates are present at a total concentration in the aqueous formulation in the range 55 to 175 mM.

16. An aqueous formulation as claimed in claim 1, wherein the one or more additional carbohydrates are present at a total concentration in the aqueous formulation in the range 60 to 135 mM.

17. An aqueous formulation as claimed in claim 1, wherein the one or more additional carbohydrates are present at a total concentration in the aqueous formulation in the range 90 to 130 mM.

18. An aqueous formulation as claimed in claim 12, wherein galactose and glucose are present in a total amount sufficient to meet carbohydrate requirements of sodium co-transport in effective rehydration, and wherein a ratio of molar concentration of galactose and glucose is in the range 0.6:1 to 1:0.6.

19. An aqueous formulation as claimed in claim 18, wherein the ratio of molar concentration of galactose and glucose is in the range 0.8:1 to 1:0.8.

20. An aqueous formulation as claimed in claim 18, wherein the ratio of molar concentration of galactose and glucose is in the range 0.9:1 to 1:0.9.

21. An aqueous formulation as claimed in claim 18, wherein the ratio of molar concentration of galactose and glucose is about 1:1.

22. An aqueous formulation as claimed in claim 12, wherein the concentration of glucose in the aqueous formulation is in the range 30 to 135 mM.

23. An aqueous formulation as claimed in claim 12, wherein the concentration of glucose in the aqueous formulation is in the range 35 to 130 mM.

24. An aqueous formulation as claimed in claim 12, wherein the concentration of glucose in the aqueous formulation is in the range 37 to 120 mM.

25. An aqueous formulation as claimed in claim 12, wherein the concentration of glucose in the aqueous formulation is in the range 40 to 100 mM.

26. An aqueous formulation as claimed in claim 12, wherein the concentration of glucose in the aqueous formulation is in the range 45 to 80 mM.

27. An aqueous formulation as claimed in claim 12, wherein the concentration of glucose is satisfied at least in part by a glucosidic oligosaccharide.

28. An aqueous formulation as claimed in claim 27, wherein the glucosidic oligosaccharide comprises maltodextrin.

29. A composition formulable in water to form an aqueous formulation as defined in claim 1, said composition comprising galactose, a source of sodium ions, optionally an additional carbohydrate, and optionally a source of additional ions.

30. A method for combatting dehydration in a subject comprising the step of:
administering to the subject an effective dose of an aqueous formulation as defined in claim 1.

31. A method as claimed in claim 30 wherein the subject is a child.
